(19) United States
(12) Patent Application Publication
(10) Pub. No.: US 2016/0199546 A1
(43) Pub. Date: Jul. 14, 2016

(54) Negative Pressure Wound Dressing

(71) Applicant: BenQ Materials Corporation, Taoyuan City (TW)

(72) Inventor: Shih-Wei CHAO, Taoyuan City (TW)

(21) Appl. No.: 14/973,746

(22) Filed: Dec. 18, 2015

(30) Foreign Application Priority Data
Jan. 8, 2015 (TW) 104200298

(51) Int. Cl. A61M 1/00; A61M 39/24

(52) U.S. Cl. CPC A61M 1/0049; A61M 1/0088; A61M 1/0025; A61M 39/24

(57) ABSTRACT

A negative pressure wound dressing including an enclosure housing and a wound contact layer is provided. The enclosure housing has an opening, the opening of the enclosure housing has a barrier layer therein to separate the enclosure housing into an exudate receptacle and a contact layer receptacle. The wound contact layer is contained in the contact layer receptacle. The exudate receptacle of the enclosure housing includes a negative pressure deliver tube connecting the exudate receptacle and a negative pressure source, and a discharging tube disposed in a vicinity of the harrier layer for discharging exudates stored in the exudate receptacle. The barrier layer includes at least one conduit with openings respectively toward the exudate receptacle and the wound contact layer for draining wound exudates from the wound contact layer to the exudate receptacle when a negative pressure is delivered from the pressure source via the negative pressure delivery tube.

### RELATED APPLICATIONS

This application claims the priority benefit of Taiwanese application serial no. 104200298, filed on Jan. 8, 2015, which is incorporated herein by reference.

### BACKGROUND

1. Field of Invention

The present invention relates to a wound dressing. Particularly, the present invention relates to a negative pressure wound dressing suitably for using in a negative pressure treatment system.

### DESCRIPTION OF RELATED ART

The human body's wound healing is an intricate process after the body tissues injured. At the very beginning, various proteins and other blood-clotting materials for wound healing will reach the wound area via blood to cause haemostatic mechanism, that is, the formation of blood clotting prevents further bleeding, and the phagocytes engulf dead cells along with bacterial, pathogens and/or debris from the damage. Then, the wound healing will be in the proliferation phase. The wound starts to repair itself and is rebuilt with new fibroblast and granulation tissue around the wound area. The granulation tissue forms and the wound contracts to close the wound. Finally, the wound area will be closed and grows with scar tissue over time.

The common wound dressing treats the wound by physical absorption of the wound exudates to keep the wound area dry. However, the common wound dressing is unable to control the humidity surrounding the wound area and the wound healing will be adversely effected due to the over dry condition around the wound area.

Negative pressure wound therapy (NPWT) is a therapeutic technique utilizing humidity control of a wound in the proliferation phase thereof to promote the intricate process of the wound healing. The negative pressure wound therapy is typically using a negative pressure source, such as a vacuum pump in connection with an airtight seal, suction member and biocompatible porous dressing to generate a negative pressure environment around the wound area to drainage the excess wound fluids and exudates, encourage the migration of the healthy tissue, maintain moisture in the surrounding tissue and increase the blood flow to accelerate the wound healing. The negative pressure wound therapy utilizing the intricate process of wound healing can effectively promote the blood flow to the area, stimulate the formation of granulation tissue and encourage the migration of healthy tissue over the wound. The negative pressure wound therapy removes the exudates from the wound tissue to inhibit the bacterial growth.

The common systems for using in negative pressure wound therapy are mainly two types, one is system with fluid storage container and the other is system with absorptive wound dressing. U.S. Patent publication No. US2011/0213287 describes a system for negative pressure treatment, a canister is disposed between the negative pressure source and the wound dressing to collect the wound fluid and exudates drained from the negative pressure treatment system. However, this negative pressure treatment system constructs with additional fluid storage container and conduits and holders, more space for this system when using this type of treatment is required, and this therapy system also restrict user's mobility. China Patent publication No. CN102046119 describes a multi-layer wound dressing for using in negative pressure wound therapy system. This multi-layer wound dressing comprises an absorptive layer and an air-permeable water barrier. The fluid and exudates sucked by negative pressure will be absorbed and contained in the absorptive layer and will not flow into the negative pressure source under the negative pressure because of the disposition of the air-permeable water barrier. However, when excess fluid is absorbed by the absorptive layer, the space in the absorptive layer will be filled with the fluid to block the airflow from the negative pressure source. Furthermore, according to the research study of the negative pressure therapy, the negative pressure should be continuously delivered for the first 48 hours and then intermittently later on for achieving preferred wound healing. However, when pressure changes, the dressing will expand due to the increased pressure. The expanded dressing will urge the wound and may damage the tissues around the wound and cause pain. Therefore, although a preferred wound healing will be achieved by intermittently applying negative pressure to the wound, this intermittent pressure application is used less. In addition, when wound is subject to apply different pressure intermittently, the dimension and the volume of the wound treatment dressing may be accordingly changed to cause the seal condition thereof with the wound area loosed and result in pressure leakage. In one case, the deformation of the wound treatment dressing due to the pressure changed may result in the fluid in the absorptive layer to flow back to the wound.

Accordingly, there is a demand of a novel negative pressure wound dressing for suitably using along with a negative pressure wound therapy system. The novel negative pressure wound dressing mentioned above is used more easily and conveniently than the one used with a canister. In addition, it is desired that the wound dressing will not be deformed under either continuously or intermittently sucking from the negative pressure. Therefore, the wound will not be pulled and the risk of the infection will be decreased.

### SUMMARY

In accordance with the above-mentioned, the present invention provides a negative pressure wound dressing for drawing the wound exudates by negative pressure from the wound to the exudate receptacle for wound exudates of the enclosure housing. The wound exudates are discharged from the exudate receptacle for wound exudates directly. Therefore, there is no need to change the dressing frequently to reduce the risk of the wound infection and to improve the convenience to the user. Furthermore, the present wound dressing can be operated under different pressure without deformation caused by the pressure change and thus, the wound will not be pulled accordingly.

The present invention provides a negative pressure wound dressing including an enclosure housing and a wound contact layer. The enclosure housing has an opening, which the opening of the enclosure housing has a barrier layer therein to separate the enclosure housing into an exudate receptacle for wound exudates and a contact layer receptacle for a wound contact layer. A wound contact layer is disposed in the contact layer receptacle. The exudate receptacle for the wound exudates further includes a negative pressure delivery tube connecting the exudate receptacle and the negative pressure source, and a discharging tube for discharging the exudates stored in the exudate receptacle disposed in the vicinity of the barrier layer. The barrier layer includes at least one conduit with openings at both ends to respectively open toward the exudate receptacle and the wound contact layer for draining wound exudates from the wound contact layer to the exudate receptacle when a negative pressure is delivered from the negative pressure source via the negative pressure delivery tube. When in operation, a negative pressure is provided by a negative pressure source via the delivery tube to the present invention, the wound exudates will be drawn from the wound contact layer through the conduit of the barrier layer to the exudate receptacle.

In a negative pressure wound dressing of one preferred embodiment of the present invention, the negative pressure tube further include an air-permeable waterproof barrier to prevent fluid from flowing into the negative pressure source.

In a negative pressure wound dressing of one preferred embodiment of the present invention, the wound contact layer includes manifold structure.

In a negative pressure wound dressing of one preferred embodiment of the present invention, the materials used for wound contact layer are selected from a group consisting of polyurethane, polyolefin, vinyl acetate, polysiloxane, fluoro-polysiloxane, fluoro-polyethylene, fluoroelastomers, styrene, butadiene and a thermoplastic elastomer.

In a negative pressure wound dressing of one preferred embodiment of the present invention, the size of the opening of the conduit toward exudate receptacle is smaller than that opened of the conduit toward the wound contact layer.

In a negative pressure wound dressing of one preferred embodiment of the present invention, the conduit further includes a check valve for preventing backflow of the exudates drawn from the exudate receptacle.

In a negative pressure wound dressing of one preferred embodiment of the present invention, the enclosure housing further includes a volume marking.

In a negative pressure wound dressing of one preferred embodiment of the present invention, the opening of the conduit toward the exudate receptacle is protruding in the space of the exudate receptacle, and the volume marking of the exudate receptacle is at the position lower than height of the conduit protruding in the exudate receptacle.

In a negative pressure wound dressing of one preferred embodiment of the present invention, the discharging tube includes a valve.

In a negative pressure wound dressing of one preferred embodiment of the present invention, the enclosure housing further includes a protective layer surrounding the peripheral edge of the enclosure housing.

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments. The above and other aspects and advantages of the present invention will become apparent from the following detailed description of the present invention taken in conjunction with the drawings.

### BRIEF DESCRIPTION OF THE DRAWINGS

FIG. 1 shows the cross-sectional prospective view of the present negative pressure wound dressing.

FIG. 2 shows the top view of the present negative pressure wound dressing.

### DETAILED DESCRIPTION

Referring now to the drawings to illustrate the embodiments of the present wound dressing. In the following embodiments of the present invention, the like elements refer to like symbols in the figures. The following description will introduce the embodiment of the above-described wound dressing. For being better understood the embodiments of the present invention, a detailed description thereof is provided. However, well-known functions or constructions may not be described in detail for brevity and/or clarity. The drawings are not necessarily to scale, emphasis instead being placed upon illustrating the principles of the invention rather than to limit and restrict of the scope of the present invention defined in the appended claim.

The advantages and features of the present invention will become better understood with reference to the following more detailed description and claims taken in conjunction with the accompanying drawings. It should be understood that the exemplary embodiments of the present invention described below may be modified in many different ways without departing from the inventive principles disclosed herein, and the scope of the present invention is therefore not limited to these particular embodiments. Rather, these embodiments are provided so that this disclosure will be thorough and complete, and will fully convey the concept of the invention to those skilled in the art by way of example and not of limitation. In the drawings, the thickness of layers, films, and regions are exaggerated for clarity. The present invention is only defined by the appended claims.

Unless otherwise defined, all terms (including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

The term “negative pressure” used herein is referred to the pressure at the wound area is lower than the ambient pressure. In most cases, the negative pressure is lower than the ambient pressure where the patient is located. Alternatively, the negative pressure is lower than a hydrostatic pressure at the wound tissue area. The negative pressure may initially generate fluid flow in the wound tissue area. As the hydrostatic pressure around the tissue area reaches the desired pressure, the flow may slow down, and the pressure then is maintained. Unless otherwise indicated, the pressure value mentioned herein is gauge pressure. Similarly, the increases in negative pressure refer to a decrease in absolute pressure and decreases in negative pressure refer to an increase in absolute pressure.

The term “wound tissue site” as used herein refers to a wound or damage located on or within any tissue, including but not limited to bone tissue, adipose tissue, muscle tissue, neural tissue, dermal tissue, vascular tissue, connective tissue, cartilage, tendons, or ligaments. The term “wound tissue site” may further refer to areas of any tissue that are not wounded or damaged, but are instead areas in which it is needs to promote the growth of tissue. For example, the negative pressure therapy may be used in certain tissue areas to grow additional tissue that may be harvested and transplanted to another location.

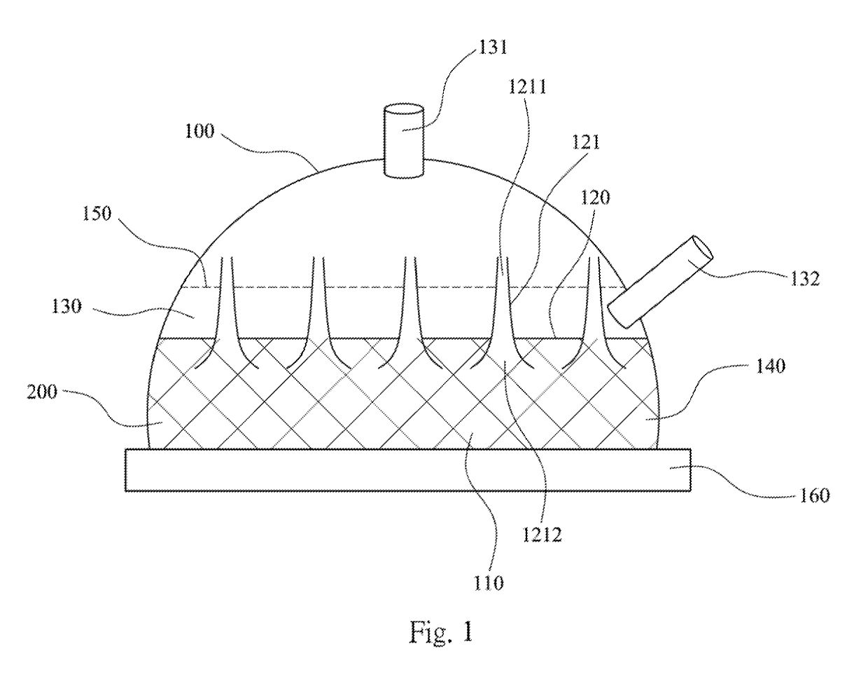

Referring now to FIG. 1, it shows the cross-sectional prospective view of the present negative pressure wound dressing. As shown in FIG. 1, the present negative pressure wound dressing includes an enclosure housing 100 and a wound contact layer 200. The enclosure housing 100 is used for maintaining the wound area in a seal condition. The enclosure housing 100 includes a barrier layer 120 to separate an opening 110 into an exudate receptacle 130 and a wound contact layer receptacle 140. The peripheral edge of the enclosure housing 100 optionally includes an adhesive layer. The adhesive layer is formed by, such as, a pressure sensitive acrylate adhesive for adhering the negative pressure wound dressing to the skin around the wound area in order to enhance the secure seal of the negative pressure wound dressing to the wound area.

Wound contact layer 200 is contained in the wound contact layer receptacle 140. In a negative pressure wound dressing of one preferred embodiment of the present invention, the wound contact layer 200 has manifold structure. The manifold structure is provided to apply negative pressure to the tissue site of wound and/or to drain or to deliver fluids therefore. The wound contact layer 200 includes a plurality of flow channels in the manifold structure, which distribute fluids provided to the tissue site of wound and removed the fluids therefrom. The flow channels are interconnected in the manifold structure. The wound contact layer 200 can be a biocompatible material which can be placed to contact with the wound tissue and distribute the negative pressure to the wound tissue. The examples of the wound contact layer 200 can be but not limited to a materials with flow channel structure, for example cellular foam (such as open-cell foam), porous tissue collections, and liquids, gels and foams that include or cure to include flow channels. In an embodiment of the present wound dressing, the wound contact layer 200 is foam with manifold structure. The foam is a porous foam including a plurality of open cells or pores connected together to be the flow paths. The porous foam can be open-celled or reticulated polyurethane foam, or flexible open-celled polymer foam, such as, for example, polyolefin, vinyl acetate, polysiloxane, fluoro-polysiloxane, fluoro-polyethylene, fluoroelastomers, styrene, butadiene or a thermoplastic elastomer.

The exudate receptacle 130 of the enclosure housing 100 includes a negative pressure delivery tube 131. The exudate receptacle 130 is fluidly communicated with the negative pressure source via the negative pressure delivery tube 131 in order to transmit a negative pressure to the wound area. The negative pressure refers to a pressure less than the ambient pressure at a tissue site that is being subjected to treatment. In most case, the negative pressure will be less than the atmospheric pressure at which the patient is located. Alternatively, the negative pressure may be less than a hydrostatic pressure associated with tissue at the tissue site. The pressure can be delivered in a constant state or in a variable state and/or delivered continuously or intermittently. In one embodiment of the present invention, negative pressure delivery tube 131 further includes an air-permeable water barrier (not shown) to block the fluids and exudates to flow into the negative pressure source. The material of the air-permeable water barrier is to prevent the fluid and exudates from flowing through the barrier but to allow air to pass through. The examples of the air-permeable water barrier can be but not limited to high vapor-permeable film or other hydrophilic polymer. In one embodiment of the present invention, the material of air-permeable water barrier can be polyvinyl alcohol, polyvinyl acetate, polyvinyl pyrrolidone, polyurethanes, polyamides, polyesters, polyacrylates and polymethacrylates, or polyacrylamide.

The barrier layer 120 includes at least one conduit 121. One of the opening 1211 of the conduit 121 is opened toward the exudate receptacle 130, and the opening 1211 is protruding in the space of the exudate receptacle 130. The other one opening 1212 of the conduit 121 is opened toward the wound contact layer receptacle 140. When a negative pressure is applied via the negative pressure delivery tube 131 from the negative pressure source, the wound fluid and exudates will be drawn via the conduit 121 from the wound contact layer 200 to the exudate receptacle 130. In one preferred embodiment of the present invention, the size of the opening 1211 of the conduit 121 opened toward the exudate receptacle 130 is smaller than that of the opening 1212 opened toward the wound contact layer 140 in order to prevent the liquid and exudates stored in the exudate receptacle 130 to backflow into the wound contact layer 200. In another preferred embodiment of the present wound dressing, the conduit 121 further includes a check valve not shown), which is disposed around the opening 1211 of the conduit 121 toward the exudate receptacle 130 for preventing the backflow of the fluid and exudates.

The exudate receptacle 130 of the enclosure housing 100 further include a discharging tube 132, The discharging tube 132 is disposed in a vicinity of the barrier layer. In detail, the discharging tube 132 is disposed at a place on the side wall but close to the harrier layer 120 of the exudate receptacle 130 for discharging the fluid and exudates in the exudate receptacle 130. In one preferred embodiment of the present invention, the discharging tube 132 further includes a valve for convenient operation. According to one embodiment of the present wound dressing, the fluid and exudates in exudate receptacle 130 can be discharged via the discharging tube 132 without to change a new wound dressing. In such a case, the risk of the wound infection caused by frequently changing the wound dressing will be decreased. In another one embodiment of the present invention, the enclosure housing 100 is made of rigid, transparent materials and optionally, marked with a volume marking 150 for user to determine the fluid volume in the exudate receptacle 130 and thus, to discharge the fluid in the exudate receptacle 130 via the discharging tube 132 as necessary. In addition, because the material for the enclosure housing 100 is transparent, it is easy to observe the exudates volume of the in the enclosure housing 100. In operation, the volume marking 150 is marked in dependent to the volume of the exudate receptacle 130 and the requirement of practice use. In one embodiment of the present invention, the volume marking 150 is at the position lower than height of the opening 1211 of the conduit 121 protruding in the space of the exudate receptacle 130 in order to prevent the exudates in the exudate receptacle 130 from backflowing to the wound contact layer via the opening 1211 of the conduit 121.

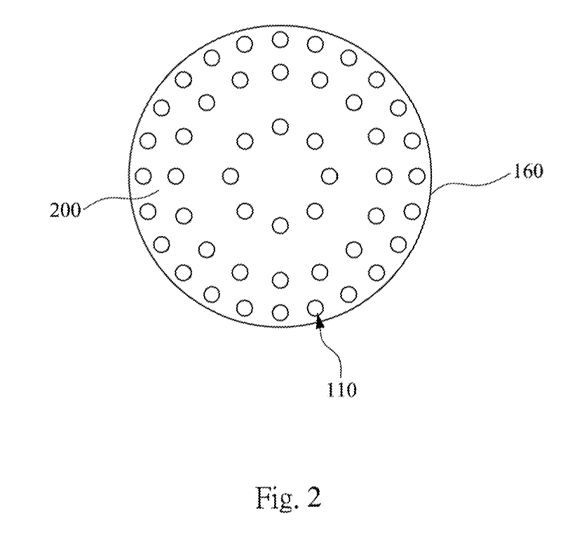

For the users' comfort, in one embodiment of the present invention, the enclosure housing 100 optionally includes a protective layer 160. The protective layer 160 is disposed at the periphery edge of the enclosure housing 100 to enhance the users' comfort when using the present wound dressing, as shown in FIG. 2. The protective layer 160 can be made of a material suitable used as buffer structure, such as closed-cell foam material, rubber of latex, in suitable thickness. The protective layer 160 is optionally to include an adhesive layer. The adhesive layer includes an acrylate adhesive for adhering the negative pressure wound dressing to the wound area for enhancing the air-tight seal of the negative pressure wound dressing.

Accordingly, when using the present negative pressure wound dressing, the wound area is sealed by the enclosure housing 100 and the negative pressure delivery tube 131 is communicated to a negative pressure source. The negative pressure is uniformly transmitted to the wound area via the wound contact layer 200. Under the negative pressure condition, the fluid and exudates on the wound area is drawn to the wound contact layer 200 and drained to the exudate receptacle 130 via the conduit 121 and stored therein. When volume of the exudates reaches the volume marking 150 on the exudate receptacle 130, the exudates can be discharged by the discharging tube 132 of the exudate receptacle 130. Therefore, the frequently change of the wound dressing is not needed.

The advantages of the present invention are as the following:

1. As comparing with the prior negative pressure wound dressing which a storage container of exudates is needed, the present invention wound dressing can be used solely without a storage container. It is more convenient for patients.

2. As comparing with the prior absorptive wound dressing, the negative pressure delivery of the present wound dressing will not be decreased even if exudates is absorbed by the wound dressing. Hence, the demand of the replacement of the present wound dressing is decreased and the risk of the wound infection will be lowered.

3. The present negative pressure wound dressing is suitable used together with various negative pressure wound therapy systems, either used with continuous or intermittent pressure supply system, the dressing will not be deformed to cause the wound-pull.

4. The enclosure housing is made of transparent material. Thus, it is easy to observe the volume of exudates in the exudate receptacle.

Accordingly, the present negative pressure wound dressing is to provide the advantages such as, convenient use, no wound-pull and lowering the risk of wound infection, to enhance the wound healing.

While the invention has been described by way of example(s) and in terms of the preferred embodiment(s), it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A negative pressure wound dressing comprising:
an enclosure housing having an opening, wherein the opening of the enclosure housing has a barrier layer therein to separate the enclosure housing into an exudate receptacle and a contact layer receptacle; and
a wound contact layer contained in the contact layer receptacle;
wherein the exudate receptacle of the enclosure housing further comprising:
a negative pressure delivery tube connecting the exudate receptacle and a negative pressure source; and
a discharging tube for discharging exudates stored in the exudate receptacle disposed in a vicinity of the barrier layer;
wherein the barrier layer comprises at least one conduit with two openings respectively toward the exudate receptacle and the wound contact layer for draining wound exudates from the wound contact layer to the exudate receptacle when a negative pressure is delivered from the negative pressure source via the negative pressure delivery tube.

2. The negative pressure wound dressing of claim 1, wherein the negative pressure delivery tube further comprising an air-permeable waterproof barrier to prevent the wound exudates from flowing into the negative pressure source.

3. The negative pressure wound dressing of claim 1, wherein the wound contact layer comprises a manifold structure.

4. The negative pressure wound dressing of claim 1, wherein a material of the wound contact layer is selected from a group consisting of polyurethane, polyolefin, vinyl acetate, polysiloxane, fluoro-polysiloxane, fluoro-polyethylene, fluoroelastomers, styrene, butadiene and a thermoplastic elastomer.

5. The negative pressure wound dressing of claim 1, wherein the size of the opening of the conduit opened toward the exudate receptacle is smaller than the size of the opening of the conduit opened toward the wound contact layer.

6. The negative pressure wound dressing of claim 1, wherein the conduit further comprises a check valve disposed around the opening of the conduit opened toward the exudate receptacle for preventing backflow of the exudates drawn.

7. The negative pressure wound dressing of claim 1, wherein the enclosure housing further comprises a volume marking.

8. The negative pressure wound dressing of claim 7, wherein the opening of the conduit toward the exudate receptacle is protruding in the space of the exudate receptacle, and the volume marking of the enclosure housing is at the position lower than a height of the opening protruding in the space of the exudate receptacle.

9. The negative pressure wound dressing of claim 1, wherein the discharging tube further comprises a valve.

10. The negative pressure wound dressing of claim 1, wherein the enclosure housing further comprises a protective layer disposed in the peripheral edge of the enclosure housing.
